(19) United States
(12) Patent Application Publication
(10) Pub. No.: US 2001/0048917 A1
(43) Pub. Date: Dec. 6, 2001

(54) SKIN EQUIVALENT AND METHODS OF FORMING AND USING SAME

(76) Inventors: WARREN HOEFFLER, SAN CARLOS, CA (US); CHARLOTTE F. NELSON, SUISUN, CA (US); CHIAOYIN KATHY WANG, PALO ALTO, CA (US)

Correspondence Address: FLEHR HOHBACH TEST, ALBRITTON & HERBERT LLP, SUITE 3400 FOUR EMBARCADERO CENTER, SAN FRANCISCO, CA, 94111

(21) Appl. No.: 09/037,191

(22) Filed: Mar. 9, 1998

(51) Int. Cl. A61K 48/00; A61K 39/00; C12N 5/06

(52) U.S. Cl. 424/93.1; 514/44; 424/277.1; 435/325; 435/1.1

(57) ABSTRACT

Methods for the formation of a mammalian skin equivalent are described herein. The method comprises mixing keratinocytes and fibroblasts. The mammalian skin equivalent is also described. The skin equivalent can be made to be normal, abnormal or aging.

### SPONSORSHIP

The U.S. Government may have certain rights in this invention pursuant to Grant No. AR 41045-01 awarded by the National Institute of Health.

### FIELD OF THE INVENTION

This invention relates to skin equivalents. More particularly, this invention relates to a mammalian, preferably human, cell sorted skin equivalent formed from a mixture of keratinocytes and fibroblasts, and methods of forming and using the same.

### BACKGROUND OF THE INVENTION

The skin, the largest mammalian organ, has a unique role in protecting organisms from the outside world. Procedures for maintaining the quality of this covering have an important place in medicine. Skin grafting can be crucial in instances where damage has been extensive, such as in severe burns, serious wounds, and ulcers. In addition, the skin is a site for the administration of pharmacological preparations, for the evaluation of toxic compounds, and for the application of cosmetics. The expediency of the need for the expansion of skin tissue and subsequent grafting of the skin to cover severely injured patients, has driven the development of methods for forming artificial skin substitutes. Attempts have also been made to provide skin models for the purpose of testing a variety of compounds and procedures.

In one approach, skin grafts have been formed from cultured autologous keratinocytes (CAK), which have been studied for applicability in treating burns, and other wounds. This method makes use of the ability to culture keratinocytes as described in Rheinwald and Green, Cell, 6:331-344 (1975), and to create graftable sheets of CAK by raising Ca²⁺ levels which induces partial differentiation. This potentially leads to the formation of desmosomes which are necessary to bind the keratinocytes together to form a sheet, but unfortunately impedes the proper formation of hemidesmosomes. Hemidesmosomes are required to connect the grafted sheet to the underlying dermis. These sheets have been applied directly to burns as described in O'Connor, et al., Lancet, 1:75-78 (1981), and ulcers as described in Leigh, et al., Br. J. Dermatol., 177:591-597 (1987), but are typically problematic due to poor adhesion to the wound, presumably due to the improper formation of hemidesmosomes. In cases where the sheet did take, the resulting graft remains fragile due to the absence of rete ridges at the dermal-epidermal junction, see, Woodley, et al., JAMA, 259:2566-2571 (1988).

The method of grafting a keratinocyte sheet is still commonly used to treat burn patients. However, not only are these grafts unlikely to take on the patient, but in cases where the graft does survive, it is only temporary. Ultimately, these grafts are entirely replaced by new skin cells, generated from the patient, not from the grafted cells. The replacement indicates that either the grafted cells are not replicating to maintain their number, or the graft is being rejected for some other reason.

More recently, researchers have focused on making grafts which contain both epidermal and dermal layers, sometimes called “full-thickness” skin. This approach is desirable since in wounds severe enough to require grafting, the dermal layer is often damaged or missing. The dermis is needed to supply nutrients and growth factors to the epidermis and is further needed for normal attachment. Many of the studies are based on work that utilized contracted collagen gels as a matrix support. See, Bell, et al., Science, 211:1042-1054 (1981). Alternatives to a pure collagen matrix have been developed including a polyglygolic acid mesh as described in Hansbrough, et al., J. Burn Care Rehabil., 15:346-53 (1994), or collagen and glycosaminoglycan matrix covered with a silastic membrane (C-GAG) as described in Burke, et al., Ann. Surg., 194:413-420 (1981). In some cases the matrix was seeded with fibroblasts giving rise to organotypic models. See, Hansbrough, et al., JAMA, 262:2125-2130 (1989); Cooper and Hansbrough, Surgery, 109:198-207 (1991); and Boyce, et al., Plast. Reconstr. Surg., 91:632-641 (1993). Naturally derived dermis, from allogenic cadaver skin has also been adapted for use with keratinocyte sheets. See, Cuono, et al., Lancet, 1:1123-1124 (1986); and Langdon, et al., J. Invest. Dermatol., 91:478-485 (1988). A variation of this technique uses lyophilized devitalized dermis from cadaver skin to support the keratinocyte sheets. See, Krejci, et al., J. Invest. Dermatol., 91:478-485 (1991); Matouskova, et al., Burn, 19:118-123 (1993); Ben-Bassat, et al., Plast. Reconstr. Surg., 89:510-520 (1990); and Medalie, et al., J. Invest. Dermotol., 107(1):121-27 (1996). In each of these cases, a support of some kind was required to reduce fragility, causing extra time and materials to be utilized.

In composite grafts, the epidermal and dermal layers are formed separately, and then sandwiched together. Since composite graphs suffer from poor adhesion, other approaches have been taken. In such an approach, an in vitro organotypical model is formed as described in Boukamp, et al., Cancer Res., 45:5582-5592 (1985), by preparing collagen gels in a silicon chamber as described in Fusenig, et al., Bull. Cancer, 65:271-280 (1978) and Fusenig, et al., J. Invest. Dermotol., 81:168s-175s (1983), and later seeding the gel with keratinocytes. As part of the characterization of the invasive properties of squamous cell carcinomas, transformed keratinocytes were in some cases tested in vivo in the silicone chambers grafted directly onto the murine muscle fascia. Boukamp, et al., Cancer Res., 45:5582-5592 (1985). These studies used only the keratinocyte cell type and therefore did not result in the recreation of skin. Although these studies used a silicon chamber, they still relied on either separate formation of the dermis and then a subsequent step of layering an epidermis on top, or use of the epidermis alone.

Therefore, it is an object of the invention to provide simple methods of forming a mammalian skin equivalent. It is also an object to provide a skin equivalent which does not rely on a synthetic or preformed support. Still further, an object of the invention is to reconstitute full-thickness human skin by allowing a mixed cell population to “cell-sort” such that the inherent cell adhesive properties of keratinocytes and dermal fibroblasts are maintained. It is a further object to provide a skin model wherein at least one cell type of the skin is abnormal. It is also an object to provide an animal model having model skin thereon which is normal or abnormal.

It is a further object of the invention to provide methods for making and using a mammalian skin equivalent. Additionally, it is an object to provide assays to identify candidates which have an effect upon skin and/or the formation of skin.

### SUMMARY OF THE INVENTION

In accordance with the foregoing objectives, the present invention comprises methods for the formation of a mammalian, preferably human, skin equivalent. The methods comprise mixing mammalian keratinocytes and dermal fibroblasts and allowing them to cell sort to form the skin equivalent. The present invention also comprises skin equivalents.

In one embodiment, the mammalian skin equivalent of the invention has a clearly defined dermis and stratified epidermis. The interface between the two layers, the basement membrane zone, is freshly formed and maintains properties of the skin that the cells are derived from. When normal cells are utilized, basal keratinocytes are formed in a density similar to that of native skin. Moreover, the basal keratinocytes express hemidesmosomes. In each embodiment described herein, no additional or artificial support is required, therefore, the epidermal layer is either in direct contact with or is adjacent to the dermal layer.

The mammalian skin equivalents and methods described herein are useful to treat individuals in need of skin or as models for testing candidate agents which may have a direct affect, or no affect, on skin or on its formation. The skin equivalents can be normal or abnormal. The skin equivalents can be formed directly on a mammal, formed and then transferred to a mammal, or maintained in tissue culture dishes indefinitely. When the skin equivalent is formed on or is transferred onto an individual, the fibroblasts and/or keratinocytes are preferably obtained from the individual.

### BRIEF DESCRIPTION OF THE DRAWINGS

FIG. 1a is a photograph depicting partial separation of human keratinocytes and fibroblasts beginning to form discrete epidermal and dermal layers from a mixed cell slurry made according to the present invention. Hematoxylin/cosin staining shows the upper epidermis in purple and the lower dermis in a lighter violet. The arrows indicate pockets of fibroblasts lingering in the epidermis prior to migration to the dermis.

FIG. 1b is a photograph depicting complete separation of human keratinocytes and fibroblasts into discrete epidermal and dermal layers from a mixed cell slurry made according to the present invention. Flaking layers at the top of the epidermis are dead differentiated squames, as would be seen in native skin.

FIG. 1c is a photograph depicting filaggrin antibody immunostaining of a skin equivalent made according to the present invention. Upper layer differentiated keratinocytes express filaggrin, and therefore stained brown.

FIG. 1d is a photograph depicting keratin 10 antibody immunostaining of a skin equivalent made according to the present invention. All keratinocytes express keratin 10 and stained brown, except the single layer of basal keratinocytes along the dermal-epidermal junction, which remained purple.

FIG. 1e is a photograph depicting keratin 14 antibody immunostaining of a skin equivalent made according to the present invention. Only basal keratinocytes express keratin 14, and therefore stained dark brown.

FIG. 1f is a photograph depicting laminin-5 antibody immunostaining of a skin equivalent made according to the present invention. Laminin-5 is a component of hemidesmosomes expressed by basal keratinocytes along the basement membrane zone (BMZ), and therefore dark brown staining is limited to the BMZ, as indicated by arrows.

FIG. 2a is a photograph depicting collagen VII antibody immunostaining of a skin equivalent made according to the present invention. Collagen VII is expressed primarily by dermal fibroblasts and is localized to the upper dermis along the BMZ, and therefore brown staining is along the BMZ as indicated by arrows.

FIG. 2b is a photograph depicting vimentin antibody immunostaining of a skin equivalent made according to the present invention. Vimentin is uniformly expressed by dermal fibroblasts, and therefore brown staining is seen ubiquitously in the dermal layer.

FIG. 2c is a photograph depicting human fibroblast specific monoclonal antibody 5B5 immunostaining of a skin equivalent made according to the present invention. This antibody recognizes human, but not mouse, fibroblasts. Fibroblasts of mouse origin are present below the human dermis, but do not stain (brown) with 5B5.

FIG. 3a is a photograph depicting normal keratinocytes used to form a skin equivalent according to the present invention. The basal keratinocytes are detected with laminin-5 antibody immunofluorescence shown localized to the BMZ.

FIG. 3b is a photograph depicting junctional epidermolysis bullosa (JEB) keratinocytes used to form a skin equivalent according to the present invention. Laminin-5 antibody does not recognize the BMZ of JEB reconstituted skin, recapitulating the disease phenotype.

FIG. 4a is a diagram of a retroviral expression vector used to express β-galactosidase in either keratinocytes or fibroblasts according to the present invention.

FIG. 4b is a photograph depicting a skin equivalent according to the present invention wherein retrovirus infected dermal fibroblasts were utilized. Blue staining limited to the dermis indicates that β-galactosidase expression localized to the reconstituted dermis.

FIG. 4c is a photograph depicting a skin equivalent according to the present invention wherein retrovirus infected keratinocytes were utilized. Blue staining is shown limited to the epidermis indicating that β-galactosidase expression localized to the reconstituted epidermis.

FIG. 5a is a photograph depicting early passage fibroblasts (passage 20) used to form a skin equivalent according to the present invention, visualized with hematoxylin/eosin.

FIG. 5b is a photograph depicting middle passage fibroblasts (passage 60) used to form a skin equivalent according to the present invention, visualized with hematoxylin/eosin. This embodiment shows some separation of epidermal and dermal layers. Arrows indicate region of dermal-epidermal shearing.

FIG. 5c is a photograph depicting late passage fibroblasts (passage 80) used to form a skin equivalent according to the present invention, visualized with hematoxylin/eosin. This embodiment shows vast separation of epidermal and dermal layers. Arrows indicate region of dermal-epidermal shearing.

FIG. 6a is a planar view of a brim 10 used according to the present invention.

FIG. 6b is a horizontal view of brim 10 used according to the present invention.

FIG. 7 is a horizontal view of a hat 18 used according to the present invention.

### DETAILED DESCRIPTION OF THE INVENTION

The mammalian skin equivalents described herein are formed from a mixture of fibroblasts and keratinocytes. The skin equivalents can be formed to have a desired phenotype including one that is normal or abnormal. Also described herein are methods of forming the mammalian skin equivalents. Applications for the skin equivalents according to the present invention are also described herein. Such applications include use for treating individuals in need of skin, use as a skin model, and use in assays to identify candidate agents which affect or have no affect on the phenotype and/or genotype of skin, or the formation of skin.

The skin equivalent of the present invention provides a number of advantages over previously disclosed attempts at providing skin equivalents. One advantage of the skin equivalent provided herein is that the cells forming the basal membrane zone maintain their inherent characteristics. For example, when normal keratinocytes are used, a basal membrane layer is formed of basal keratinocytes which express hemidesmosomes. The hemidesmosomes serve to tightly connect the epidermal layer to the dermal layer. Additionally, desmosomes are formed. Desmosomes hold the keratinoctyes to each other. Therefore, the skin equivalent can be formed to have an essentially intact basal membrane zone which is naturally resistant to shearing and disruption. Additionally, the retention of the basal character of the keratinocytes provides for the skin equivalent to be regenerative. Therefore, the skin equivalent will regenerate to maintain the characteristics of the cells from which it was formed.

Another aspect of this invention is that it provides a skin equivalent which can be formed, maintained and used essentially in tissue culture thereby circumventing the need for using animals to test products which may be harmful to the skin. For example, companies which are in the practice of testing products on animals to determine if the products cause skin discoloration, aggravation, etc., can test the products on the skin equivalent provided herein wherein the skin equivalent is formed and maintained in tissue culture.

Another aspect of the present invention is that since the cells from which the skin equivalent is formed maintain their inherent characteristics, the skin equivalents can also be formed to have a variety of different phenotypes. For example, the skin equivalent can be formed with cells which have been treated or manipulated prior to formation of the skin equivalent. Therefore, the skin equivalent can be formed with, e.g., genetically engineered cells, aging cells, or cells obtained from an individual with a skin disorder such as psoriasis, rosacea, or junctional epidermolysis bullosa (JEB). In these cases the skin equivalent that is formed will have the phenotype and/or genotype of these cells. Such skin equivalents are useful for models to test treatments, or in some cases to provide “skin patches”. For example, a skin patch can be formed with cells which are genetically engineered to provide a particular substance to the skin or body, and then grafted onto an individual. Preferably, the skin patch is inducible such that the substances to be delivered, i.e., hormones, insulin, etc., can be induced as desired.

Moreover, because the skin equivalent provided herein is initially formed from a slurry, not only can the cells be treated or manipulated prior to forming the skin equivalent, but substances can be added to the slurry to be incorporated into the skin at its initial formation, rather than having to be incorporated after the skin is already formed. For example, in addition to fibroblasts and keratinocytes, the slurry can comprise various cell types, e.g., melanocytes, hair follicular stem cells or epithelial cells.

As seen from the foregoing, the present invention is useful in a number of applications. Additional applications are described in detail below and will further become apparent as particular embodiments are described.

In one of the preferred embodiments provided herein, the mammalian skin equivalent comprises discrete epidermal and dermal layers wherein the dermal layer comprises fibroblasts and the epidermal layer comprises differentiated keratinocytes. In this embodiment, the epidermal and dermal layers are in direct contact with each other. In an alternative embodiment, there are gaps between the epidermal and dermal layers, e.g., as seen in conditions such as aging.

By “mammalian”, it is meant that the skin equivalents described herein can serve as a skin equivalent for any mammal. Humans are the preferred mammal. However, the invention can be practiced with other mammals such as non-human primates and members of the bovine, ovine, porcine, equinine, canine and feline species as well as rodents such as mice, rats and guinea pigs and members of the lagomorph family including rabbit. The particular mammalian skin equivalent which will be formed will be dependent on the source of the keratinocytes and fibroblasts, e.g., when human keratinocytes and fibroblasts are used to form the skin equivalent, a human skin equivalent is formed.

In the case of a particular species, it is preferred that the keratinocytes and fibroblasts come from the same species. When used as a skin graft, it is preferred that the cells be derived from the individual of the species to be treated. However, in some instances it may be desirable to make a heterogeneous skin equivalent, i.e., with cells derived from different individuals or with cells derived from different species. For example, porcine tissue may be a potential universal donor for use in human applications. Therefore, porcine keratinocytes and/or fibroblasts may be useful in preparing heterogenous skin equivalents (e.g., human/porcine skin equivalent). Alternatively, homogenous skin equivalents (porcine keratinocytes and porcine fibroblasts) may be prepared for use in a heterologous system, e.g., humans. In a preferred embodiment, a human skin equivalent is formed on or transferred onto a laboratory animal to form an animal model having a human skin equivalent thereon.

In a preferred embodiment, the epidermal layer comprises at least basal keratinocytes, i.e., keratinocytes which are not differentiated. The epidermal layer may further comprise partially differentiated keratinocytes as well as fully differentiated keratinocytes. In normal native skin there is, in general, a transition from undifferentiated basal keratinocytes to fully differentiated keratinocytes as one progresses from the dermal-epidermal junction where the basal keratinocytes are localized. Surprisingly, it has been observed that the skin equivalent of the present invention also displays this morphology, in particular, the presence of basal keratinocytes. This is a significant feature which has not been demonstrated in the prior art.

In native skin, basal keratinocytes express hemidesmosomes which serve to help secure the epidermal and dermal layers together. Basal keratinocytes, which are also known to be stem cells, also serve to regenerate the skin. In the skin equivalent of the present invention, the basal keratinocytes are present and thus can serve these functions. Thus, the skin equivalent containing such basal keratinocytes is capable of regeneration which is especially useful in skin graft applications. Other distinctions between basal keratinocytes and differentiated keratinocytes are that both E- and P- cadherins are present in epidermal keratinocytes along the basal membrane zone (BMZ), but keratinocytes which are differentiated and located away from the BMZ only express E-cadherin. In this regard, the presence of hemidesmosomes at the junction of the dermal and epidermal layers of the skin equivalent of the invention has been demonstrated herein by the use of labeled antibodies against laminin-5 which is a component of hemidesmosomes.

In one embodiment, the basal keratinocytes of the epidermal layer are aligned in a layer in direct contact with the dermal layer, serving as the boundary between the differentiated keratinocytes and the fibroblasts. In an alternative embodiment, there are gaps between the basal keratinocytes and the dermal layer. Still further, there may be gaps between the basal keratinoctyes and other basal keratinocytes, leaving gaps between the differentiated keratinocytes and the dermal layer. In these latter embodiments where there are gaps between the basal or differentiated keratinocytes and the dermal layer, the dermal and epidermal layers are not uniformly in contact with one another, but are adjacent to each other. They are adjacent in that there is generally fluid, but substantially no other intervening materials such as layers of cells, collagen, matrices or other supports between the dermal and epidermal layers.

In general, the keratinocytes and fibroblasts used in making the mammalian skin equivalent are obtained from primary sources (i.e., an individual) or from a cell line maintained in tissue culture. In one embodiment the fibroblasts and keratinocytes are from the same individual or cell line. In an alternative embodiment the fibroblasts and keratinocytes are from different individuals or cell lines and therefore have different genotypes. For use as a skin graft, the fibroblasts and/or keratinocytes are autologous, however, the fibroblasts and/or keratinocytes can also be allogenic, xenogenic or any mixture thereof. In a preferred embodiment, the fibroblasts are autologous. The cells may be treated or modified so as to be resistant to rejection by the host. The keratinocytes and/or fibroblasts can be immortalized as previously described. Briefly, cells can be immortalized, for example, by transfection with p18ccB containing a subgenomic fragment of HPV-18 encoding intact open rending frames of F6 and E7 as described in Barbosa and Schlegel, Oncogene, 4:1529-1532 (1989); and Villa and Schlegel, Virology, 181:374 (1991).

In some embodiments, e.g., for the study of aging skin, the fibroblasts and/or keratinocytes can be senescencing. Senescencing cells can be formed by passing the cells over and over. The number of passages will be dependent on the cell type. In each case, the skilled artisan will recognize when the cells are senescencing. Alternatively, the senescencing cells can be derived from primary sources wherein the individual shows the symptoms of aging skin such as looseness, dryness and/or wrinkles.

The phenotype of the skin equivalent will be dependent on the cells which are used to form the skin equivalent. In one embodiment the skin equivalent, which is defined herein as non-native, resembles normal native skin. The terms “native skin” and “natural skin” are used interchangeably herein and refer to the skin which an individual is born with. Normal refers to skin which is healthy and not damaged, injured or afflicted with disease. Normal includes phenotypes of different pigments, age, thickness and textures as would be seen in native skin. In general, the skin equivalent of the invention when made from normal keratinocytes and fibroblasts, will have the primary characteristics of “normal” skin, i.e., dermal and epidermal layers joined by a basal membrane zone. However, the skin equivalent lacks at least one characteristic of normal skin, distinguishing it therefrom. For example, in one embodiment, the skin equivalent of the present invention lacks at least one of hair follicles, melanoctyes, sweat glands and nerve endings. In another embodiment, the phenotype is normal, but at least one of the keratinoctyes and/or fibroblasts has a different genotype than the other cells of that cell type.

In one of the embodiments, the skin equivalent has the phenotype of aged skin. Aged skin is defined as having the characteristics of being loose, dry and/or wrinkled. Aged skin is also identified by a decrease in the thickness of the dermis, a disorganization of collagen bundles and elastin fibrils in the dermis, a decreased rate of keratinocyte turnover and/or increased fragility of the skin along with dermal-epidermal junction. The embodiment displaying the phenotype of aged skin is made by utilizing skin cells derived from aging individuals or by using senescencing cells as described herein. This embodiment can be distinguished from native aging skin by its lack of at least component normally contained in native aging skin, i.e., hair follicles, melanoctyes, sweat glands and nerve endings. Alternatively, this embodiment can be formed wherein at least one of the keratinoctyes and/or fibroblasts has a different genotype than the other cells of that cell type.

In an another embodiment, the phenotype and/or genotype of the skin equivalent is abnormal. Abnormal phenotypes or genotypes include skin which is diseased or damaged, either temporarily or permanently, Examples include diseases or afflictions such as psoriasis, cancer, acne, radiation damage, heat damage, junctional epidermolysis bullosa (JEB), scleroderma, xeroderma pigmentosus, and rosacea.

The fibroblasts and/or keratinocytes which are used in the present invention can be normal or abnormal. The fibroblasts and/or keratinoctyes can be naturally occurring or modified. Such cells can be modified by treatment with various compounds to induce changes in the phenotype or genotype. Alternatively, such cells may be genetically engineered cells. Genetically engineered is defined as a man-made directed alteration to the nucleic acid content of the cell. Therefore, genetically engineered cells include cells containing an insertion, deletion, and/or substitution of one or more nucleotides in the genome of a cell as well as alterations including the introduction of self replicating extrachromosomal nucleic acids inserted into the cell. Genetically engineered cells also include those wherein transcription of one or more genes has been altered, e.g. increased or inhibited.

Abnormal fibroblasts and/or keratinocytes include those that are over-exposed to or damaged by UV rays or toxic agents or which have been genetically engineered or have congenital defects. Examples include basal keratinocytes which are defective in expressing hemidesmosomes or laminin-5, e.g., JEB cells, cancerous cells, and radiation damaged cells. Other examples include cells which do not express one or more components generally expressed in normal native skin cells. Specific examples include keratinocytes lacking normal expression of filaggrin, laminin 5, hemidesmosomes, BP180, BP230, keratin—including keratin 10 and 14, desmosomes, keratohyalin and E- and P-cadherins. Still other examples include fibroblasts lacking normal expression of collagen—including collagen VII and IV, elastin, vimentin and the antigen localized by 5B5 antibody.

Combinations of cells with different phenotypes/genotypes can also be used. Thus, two or more different keratinocytes and/or fibroblasts can be used in the invention to determine the affect that one cell type confers on skin formation or survival. Each layer of the skin equivalent, therefore, need not be derived from a single source.

In one embodiment, the fibroblasts and/or keratinocytes are transformed or transfected with a nucleic acid which serves to express a gene product not otherwise expressed in the cell type or which inhibits the expression of a specific gene or genes in the cell. The latter is preferably carried out using well known techniques in the art such as by the use of antisense molecules. Examples of gene products that the cells can be manipulated to express or inhibit include those which are normally expressed by keratinocytes and fibroblasts and described herein, as well as antibodies, anti-cancer agents, anti-aging agents, insulin, clotting factors, vitamins, telomerase, nutrients, hormones, steroids, pigments, chemokines and cytokines. In a preferred embodiment, the expression of the nucleic acid is inducible such that an individual can, for example, rub a cream comprising the inducing agent on the skin equivalent comprising the genetically engineered cells so as to induce expression and/or secretion as desired.

The nucleic acid (e.g., cDNA or genomic DNA) encoding the desired gene product may be inserted into a replicable vector for expression. Various vectors are publicly available. The vector may, for example, be in the form of a plasmid, cosmid, viral particle, adenovirus, artificial chromosome or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

In another embodiment, the mammalian skin equivalent is regenerative. In this embodiment, stem cells survive during in the formation of the skin equivalent.

In one embodiment, the skin equivalent has at least one of the components of native skin such as melanocytes, hair follicles, sweat glands and nerve endings. In the preferred embodiment, the skin equivalent is distinguished from normal native skin by its lack of at least one of these components. In some embodiments displaying abnormal phenotypes or having at least one cell with an altered genotype, the skin equivalent can include all of these components.

In some embodiments it may be desirable to add other components to the skin equivalent. Components include myoepithelial cells, duct cells, secretory cells, alveolar cells, langerhans cells, Merkel cells, adhesions, and mammary glands. In one embodiment, bioactive molecules are included. Preferably, these components or the genes encoding therefor are included in the keratinocyte/fibroblast slurry. In a preferred embodiment, the bioactive molecules are growth factors. Examples of growth factors include heparin binding growth factor (hbgf), transforming growth factor alpha or beta (TGFβ), alpha fibroblastic growth factor (FGF), epidermal growth factor (TGF) and vascular endothelium growth factor (VEGF), some of which are also angiogenic factors. In some embodiments it may be desirable to incorporate factors such as nerve growth factor (NGF) or muscle morphogenic factor (MMP). Steroidal anti-inflammatories can also be used to decrease inflammation. The components which are added can be the components themselves or the gene expressing the component or increasing or causing expression of the component. Combinations of these components can also be added. Many of the bioactive factors are contained in wound fluid. Wound fluid or the components of wound fluid can also be included.

Combinations of fibroblasts and/or keratinocytes can be used in the present invention. For example, the keratinocytes can be a mixture of autologous and allogenic keratinocytes, wherein the autologous keratinocytes are diseased and aging, and the allogenic keratinocytes are genetically engineered.

The present invention provides procedures for forming mammalian skin equivalents which are simpler and quicker than methodologies requiring separate formation of the dermis and epidermis. In a preferred embodiment, sorting-out of epidermal cells from a mixed cell population provides for localization of undifferentiated keratinocytes to the basement membrane zone in a tightly packed linear array, analogous to the morphology seen in normal skin. This contrasts with the morphology of the keratinocytes in preformed dermal sheets shown in prior studies wherein the keratinocytes are partially differentiated, including at the basement membrane, precluding keratinocytes which are basal (undifferentiated) in character.

In one embodiment, cells are isolated from autologous or allogenic excision of tissue or from a cell line, then grown in cell culture. To obtain cells from individuals, the area to be biopsied can be locally anesthetized with a small amount of lidocaine injected subcutaneously. Alternatively, a small patch of lidocaine jelly can be applied over the area to be biopsied and left in place for a period of 5 to 20 minutes, prior to obtaining biopsy specimen. The biopsy can be obtained with the use of a biopsy needle, a rapid action needle which makes the procedure extremely simple and almost painless. A 4-6 mm punch biopsy can be used. Relative contribution of cell type can be obtained by mixing either keratinocytes or fibroblasts derived from the individual with the normal complementary cell type in reconstitutions. This small biopsy core of tissue can then be transferred to media such as SFM or KGM (Clonetics Corp.).

Cells are dissociated using standard techniques, such as treatment with collagenase or trypsin. Alternatively, the tissue biopsy can be minced and the cells dispersed in a culture plate with any of the routinely used medias. After cell expansion within the culture plate, the cells can be passaged utilizing the usual techniques until an adequate number of cells is achieved. The fibroblasts and/or keratinocytes can be maintained and/or proliferated in culture in standard cell culture dishes, until utilized.

The fibroblasts and/or keratinocytes can be transfected or transformed with expression or cloning vectors and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity and life of cell cultures can be found in The Keratinocvte Handbook, Leigh, Lane and Watt, ed. (Cambridge University Press, 1994).

Methods of transformation and transfection are known to the ordinarily skilled artisan. Such methods include, for example, lipofection (BRL), microinjection and electroporation. For various techniques for transforming mammalian cells, see e.g., Keown, et al., Methods in Enzymology, 185:527-537 (1990) and Mansour, et al., Nature, 336:348-352 (1988).

To form the mammalian skin equivalent directly on the individual, the keratinocytes and fibroblasts are thoroughly mixed to form a slurry which is then added to a “chamber” implanted on the individual. The chamber can be a commercially available inert silicone bubble chamber as described in Fusenig, et al., Bull. Cancer, 65:271-280 (1978), Fusenig, et al., J. Invest. Dermotol., 81:168s-175s (1983), Navsaria, et al., Burns, 20(1):S57-S60 (1994), and as commercially available from, for example, Renner GMBH, Germany. The chambers are commonly used in conventional human skin reconstitutions, where a preformed dermal layer provides support for the subsequent addition of keratinocytes. The chamber contains a brim, or lower chamber, which serves as a physical barrier to contain the cell slurry and prevent overgrowth of tissue onto the graft area. The second component is the hat, or upper chamber, that provides a moist environment to allow the survival of added cells, yet is not entirely encapsulated. Other devices which hold the cell slurry in place as well as provide a moist environment can be used. Preferably, the chamber is flexible so as to be able to conform to an area of an individual's body.

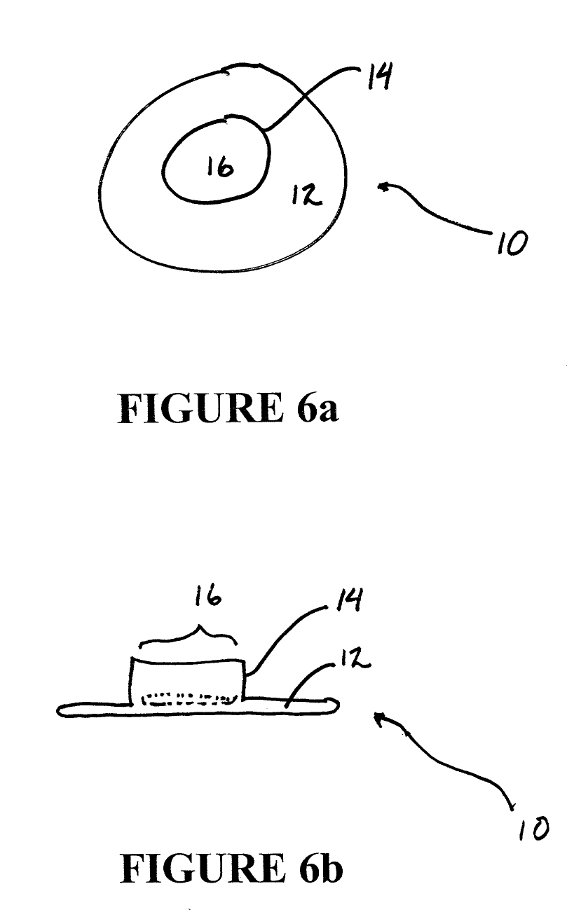

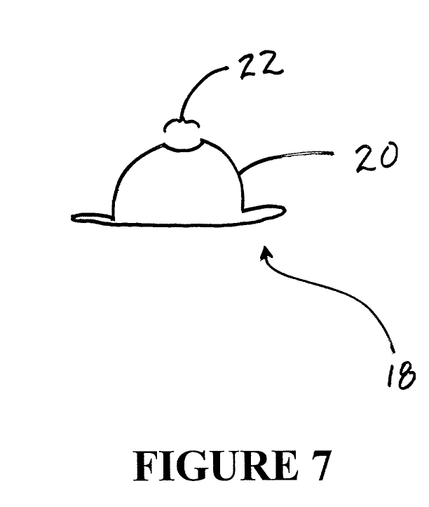

A preferred embodiment of the brim of the chamber is shown in FIG. 6a, which shows a planar view and FIG. 6b, which shows a horizontal view. In FIG. 6a, brim 10 is shown with surface 12 which preferably tucks in under an individual's remaining skin. FIG. 6b shows that rim 14 rises generally perpendicular to surface 12 to keep the cell slurry in place. The cell slurry is placed in orifice 16, directly onto the, for example, muscle fascia of the individual. FIG. 7 shows a horizontal view of the hat 18 which fits onto brim 10 to complete the chamber. Hat 18 has domed cylindrical wall 20 to provide moisture and orifice 22. While the chamber is shown in FIGS. 6 and 7 as comprising two components, the chamber can be formed in a variety of alternative embodiments, so long as the chamber holds the cell slurry in place directly on the individual and provides a moist environment.

In an alternative embodiment, the slurry of keratinocytes and fibroblasts are maintained in a cell culture dish where the cell sorting phenomenon takes place until the skin equivalent is formed. In one embodiment, wound fluid, preferably in about an equal volume, is added to the slurry. The skin equivalent is maintained for use in assays or until transfer to an individual. In another embodiment, one or more components of wound fluid is added to the slurry.

In another embodiment, the culture dish has living tissue in the bottom of the dish having a chamber implanted thereon. When the chamber is implanted thereon, the tissue is wounded by incision thereto. The slurry is added to the chamber before the wound is healed. When the skin equivalent is formed, the skin can be surgically removed from the tissue in the dish, and grafted onto an individual using standard techniques. Alternatively, the skin equivalent can be maintained in the dish where it can be used to test the effects of products administered thereto. In yet another embodiment, the skin equivalent is formed on one individual and then removed and grafted onto another individual.

In the embodiments wherein the skin equivalent is formed on or is to be transferred onto an individual that is not severely immunodeficient, and wherein non-autologous fibroblasts and/or keratinocytes are utilized, the individual can be treated so as to help inhibit rejection of the skin equivalent. Alternatively, the skin equivalent is formed so as to be resistant to rejection.

In all of the methodologies described herein, substances other than keratinocytes and fibroblasts can be added before, during or after formation of the skin equivalent. In some embodiments it may be desirable to add bioactive molecules. In one embodiment, wound fluid is added to the mixture before or during formation of the skin equivalent. Factors such as hbgf, TGF, VEGF, MMP, NGF and anti-inflammatories are known to those skilled in the art and are available commercially or described in the literature.

Dosages are calculated based on in vitro release studies in-cell culture. Preferably, the bioactive factors are incorporated to be between one and 30% by weight, although the factors can be incorporated to a weight percentage between 0.01 and 30% weight percentage. In one embodiment the bioactive molecules are prepared in time release polymers.

Wherein hair follicles, melanocytes, sweat glands or nerve endings are added, they are preferably added during formation of the skin, however, they can be implanted after formation of the skin.

Use of the mammalian skin equivalents described includes the creation of animal models or artificially maintained skin models for the study of dermatology, and in particular, dermatological diseases and conditions such as sunburn and aging. Typically in genetic diseases only one cell type expresses the defective gene, therefore, accurate modeling of the disease can be achieved by mixing normal cells with the individual cell type that expresses the defective gene. Animal models can be formed wherein the model skin is from the same or a different animal type. For example, human skin can be formed on a rodent.

In other applications, the skin equivalent is provided to individuals in need thereof. In particular, individuals having diseases, disorders, injuries, wounds, burns, scars, augmentations including breast augmentations, transplantations or unsightliness due to any reason, may be in need of a skin equivalent according to the present invention.

In still other applications the skin equivalent is used as a delivery vehicle (also termed a skin patch, herein) for substances to be delivered to an individual such as drugs, hormones, insulin and steroids.

The skin equivalent can be provided to an individual to replace large or small segments of skin. In the case of a skin patch wherein substances are provided to the body via the skin patch, a small area of the skin equivalent can be provided to an individual.

In one embodiment, an assay for identifying candidate agent(s) having an affect or no affect on skin is provided. In this embodiment, a skin equivalent is provided according to the methodologies described herein. The skin equivalent is then contacted with a candidate agent and it is determined whether the candidate agent has an affect on the skin equivalent. When the skin equivalent is formed to have a normal phenotype, this assay is useful to identify agents which have an adverse effect on the phenotype, which change the phenotype, or which do not affect the phenotype. Similarly, when the skin equivalent is formed to have a phenotype which is abnormal or which shows signs of aging or overexposure to the sun, this assay is useful to identify agents which do and do not modify such a phenotype.

In an alternative embodiment, an assay is provided for identifying a candidate agent having an affect or which has no affect on the formation, phenotype or genotype of skin. In this embodiment, the candidate agent is added to the keratinocytes and/or fibroblasts before the skin equivalent is formed into discrete layers.

In the assays described herein, the candidate agent(s) can be any agent which may modify the phenotype, genotype or formation of the skin equivalent. The candidate agent can be in the form of a gene administered to the keratinocytes and/or fibroblasts, or in the form of the gene product. Candidate agents include any cosmetic, therapeutic or pharmaceutical including anti-cancer and anti-aging agents--including telomerase, genes, oligos, peptides, radiation, and any other toxic or non-toxic agent. Other candidate agents include hbgf, TGFβ, FGF, TGF, VEGF, NGF MMP as well as other factors. Candidate agents also include those components normally expressed in by skin cells as described herein, or inhibitors of those components. Candidate agents further include peptides and factors isolated from wound fluid. In one embodiment, wound fluid is added wherein one component is missing to determine the affect of the missing component. Candidate agents also include adhesions such as cadherins. In one embodiment, the expression of E- or P-cadherins is inhibited or increased and the affect determined.

Identification of candidate agents which have an affect on the phenotype, genotype or formation of skin equivalents are useful for diagnostic and therapeutic purposes in dermatology. In particular, candidate agents which are identified as having beneficial effects can be formed into cosmetic or pharmaceutical formulations and then administered. Administration can be topically, subcutaneously, or by injection to the skin or skin cells. Alternatively, the candidate agents identified as effective agents can be used to treat the cells ex vivo and then the treated cells can be administered to the individual in the form of a slurry or an already formed skin equivalent. In one embodiment, the individual's own cells can be treated and replaced.

Similarly, agents which are identified as harmful to the phenotype, genotype or formation of skin equivalents are also useful for diagnostic and therapeutic purposes in dermatology. In particular, they are useful to identify cures and treatments to prevent or reverse the affect of such an agent.

Moreover, agents which are identified as having no affect to the phenotype, genotype or formation of skin equivalents are useful in formulations for cosmetics and pharmaceuticals which will be safe for use on skin.

The following examples are meant to further illustrate the invention, and are not meant to be limiting in any way to the spirit and scope of the invention. All references are incorporated herein in their entirety.

### SPECIFIC EXAMPLES

### Example 1

### Cell Sorted Skin Equivalent

Cell Culture

Keratinocytes were cultured from neonatal foreskin and patient biopsies and used for transfection by lipofectin (BRL) after one or two passages. Cells were grown in KGM (Clonetics Corp.) and transfected with p18ccB containing a subgenomic fragment of HPV-18 encoding intact open reading frames of E6 and E7. Barbosa and Schlegel, Oncogene, 4:1529-1532 (1989); and Villa and Schlegel, Virology, 181:374 (1991). These genes are sufficient for inducing the immortalization of keratinocytes. At 2 days post-transfection, selection for G418 resistance (100 mg/ml) was applied for a total of 10 days. Individual colonies were cloned and passaged once a week at a split ratio of 1:10. The cell lines that survived more than 20 passages in culture were considered immortalized and used for study.

Grafting

A mixed cell slurry containing approximately 6×10⁶ keratinocytes and 6-8×10⁶ dermal fibroblasts was prepared for each mouse to be grafted. Typically two 100 mm dishes of confluent keratinocytes and four dishes of confluent dermal fibroblasts yielded the needed numbers of cells. Keratinocytes were trypsinized in 0.1% trypsin, and fibroblasts in 0.25% trypsin, and later neutralized with PBS/10% calf serum. The two cell types are mixed in serum free medium (SFM, Gibco), placed in a 15 ml polystyrene conical centrifuge tubes (Falcon) and centrifuged at low speed in a clinical centrifuge for approximately 5 minutes. Excess medium was removed by aspiration, and the cell pellets were stored on ice until use.

Silicone chambers implanted on the backs of severe combined immunodeficient (SCID) mice were used as in vivo chambers for the development of reconstituted human skin. Briefly, after anesthetizing the host mice, a 1 cm diameter circle of upper back skin is removed using curved surgical scissors. The brim of the silicone chamber (CRD culture chambers, Renner, Dannstadt, Germany) is placed under the edge of the skin around the perimeter of the surgical wound incision, and clipped in place using wound clips at opposite sides of the brim. The second piece of the chamber termed the hat is fitted securely over the brim. The mixed keratinocyte-fibroblast cell suspension is transferred into the chamber directly onto the mouse muscle fascia through the 3 mm hole in the crown of the silicone hat. In the days following grafting wound fluid builds up in the chamber. After one week the silicone hat is removed and the wound allowed to dry for an additional week before biopsies are taken.

Immunohistochemistry

To examine the cell sorted skin equivalents cryosections were prepared onto slides. Briefly, fixation was with −20° C. acetone for 10 minutes. Samples were rehydrated with 5 successive PBS washes. Blocking was conducted with mouse IgG diluted 1:400 (Jackson InmunoResearch). A biotin/avidin-peroxidase conjugation system was used. 50 μl of an appropriate dilution of the primary antibody was incubated with the sample for 1 hour at room temperature, followed by 3 washes with PBS. The secondary antibody was anti-mouse Ig, horseradish peroxidase (Amersham) and was incubated with the samples for 40 minutes. After 3 washes with PBS the samples were developed with an insoluble peroxidase substrate (Sigma, St. Louis, Mo.) for 20-30 minutes.

For immunofluorescence detection of laminin-5, cryosections were prepared and subjected to a FITC-labelled secondary antibody staining method. In brief, after blocking with goat serum for 20 minutes at 37° C., the sections were incubated with primary antibody for 1 hour at 37° C. Slides were washed between each step in three shifts of PBS for a total of 15 minutes. All incubations were conducted for 30 minutes at room temperature, except where otherwise stated above. The slides were lightly counterstained with hematoxylin, dehydrated, and mounted. Negative controls consisted of a non-immune rabbit IgG applied to adjacent sections at the same concentration.

Extended Incubation

The incubation period of the reconstituted skin was extended to 2 weeks. Due to the accumulation of wound fluid in the chambers the top part of the chambers (hats) were removed after 7 days to allow drying of the reconstituted skin. Exposure of keratinocytes to an air-liquid interface induced the keratinocytes to differentiate in earlier raft models. In the cell sorted skin equivalent, the reconstituting skin was exposed to the air after the first week to achieve a clean full-thickness reconstitution with normal differentiation of the epidermal layers. Part of the tissue was imbedded in paraffin for histology, while part of the biopsy was snap frozen in liquid nitrogen, cryosectioned, and used for immuno-histochemistry. A panel of antibodies specific for standard markers of both dermal fibroblasts and keratinocytes were used. In many cases these reagents are human specific, and will not recognize the antigen from mouse cells, excluding the possibility that mouse cells and not human cells are forming the reconstituted skin.

### Results

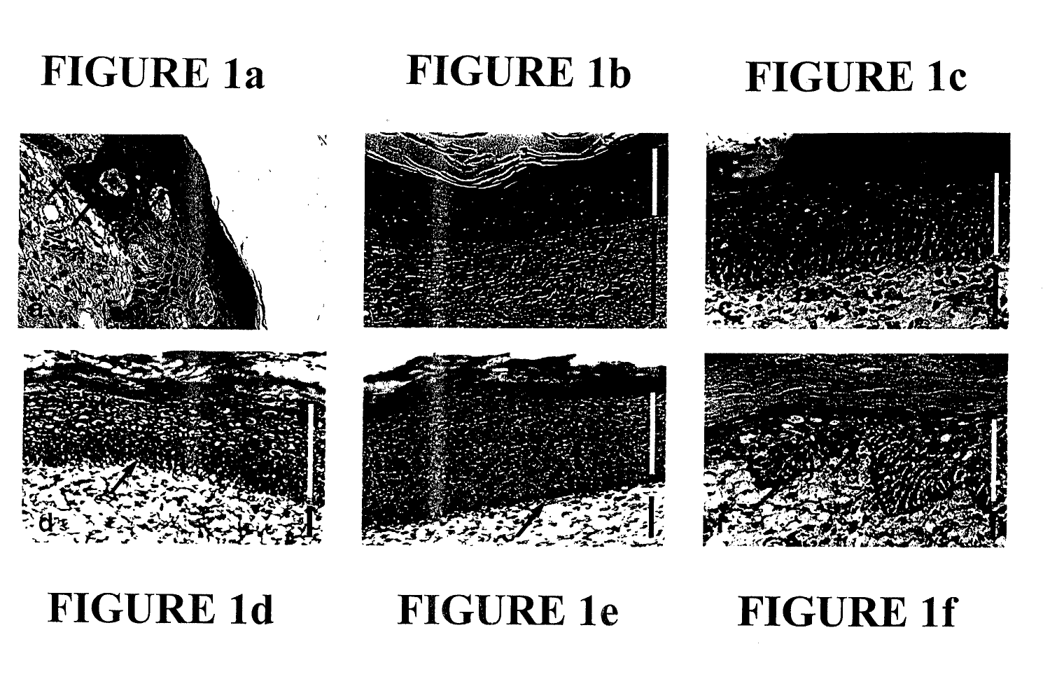

The mixture of approximately 6×10⁶ primary human keratinocytes and 6×10⁶ primary human fibroblasts were added as a thoroughly mixed cell slurry through a hole in the top chamber implanted on a SCID mouse. During the following several days mouse wound fluid was found to accumulate in the chambers. A biopsy was taken at 7 days post grafting and analyzed by hemotoxylin/eosin staining of a paraffin embedded thin section (FIG. 1a). The section showed great similarity to the morphology of natural full-thickness human skin, including epidermal layer at the top, and dermal layer at the bottom. Starting from the top of the section, a clear stratum corneum is seen as wispy layers. The obvious departure from natural skin is the inclusion of pockets of dermal fibroblasts in the epidermis, as indicated by arrows (FIG. 1a).

It is believed that the inclusion bodies represent an initial incomplete separation of epidermal keratinocytes from fibroblasts. The fibroblast body at the lower left seems to have formed a connection to the dermis, and perhaps these cells would later empty out into the dermis. The clumping of keratinocytes thus appears to be a preliminary step in sorting-out from fibroblasts. The results showed that two distinct layers were later formed.

Hematoxylin/eosin (H/E) staining of a thin-section (5 μm) from a biopsy taken at 2 weeks again showed a morphology of the reconstituted skin very similar to natural skin (FIG. 1b), and generally without pockets of incomplete sorting of keratinocytes and fibroblasts. Again, a differentiated cornified layer is seen at the top. The epidermis, stained dark purple in this figure, appears normal. The dermal-epidermal junction is very clean, with the population of dermal fibroblasts distinctly separated from the keratinocytes in the epidermis. (In this and subsequent figures, the location of the reconstituted human epidermis is indicated by a vertical white bar present on the right side, and the human dermis by the black bar. The region between the bars defines the basement membrane zone, the normal interface of the two skin layers).

The dermis is more tightly packed with fibroblasts than the normal morphology, characterized by a sparser distribution of fibroblasts contained in an extensive extracellular matrix (ECM). Since the ECM is slowly deposited by fibroblasts over time, it is believed that with increased time greater deposition of ECM would occur. The fact that a mixed population of keratinocytes and fibroblasts were added as a slurry to the chambers and cleanly separated into two separate populations within 2 weeks shows that these cells have the necessary signaling and motility to migrate apart, thereby demonstrating the phenomenon known as cell-sorting.

Immunohistochemistry was conducted with antibody reagents specific for keratinocyte markers to document the normal differentiation of the epidermis in the reconstituted model and to show that the keratinocytes are human derived and not murine in origin. Peroxidase staining using an antibody specific for human filaggrin, a late stage differentiation marker for keratinocytes, is shown FIG. 1c). Characteristic brown staining is seen in the upper layer of keratinocytes, representing human keratinocytes (not mouse) in the latter stages of differentiation. The lower layers closer to the basement membrane zone (BMZ) are not stained brown by the peroxidase but remain purple, indicating that they do not express filaggrin. This pattern is characteristic of natural skin. Likewise, another antibody against a late stage differentiation marker, keratin 10, was used (FIG. 1d). Staining is observed in the differentiated layers, with a single cell layer of unstained keratinocytes remaining along the BMZ (arrow). This pattern is also characteristic of a natural differentiated epidermis. The single layer of keratinocytes along the BMZ would be expected to stain with keratin 14 antibody, since keratin 14 is made exclusively by basal keratinocytes. Staining with this antibody did, in fact, show dark brown staining along the single layer of keratinocytes along the BMZ (FIG. 1e, arrow). Thus, these markers suggest that normal differentiation is occurring in the epidermis of the reconstituted skin, and that the cells are human in origin.

To establish that the dermal-epidermal junction of the reconstitution indeed expresses components uniquely expressed along the BMZ, a monoclonal antibody that recognizes the human laminin-5 β3 chain, but has no cross reactivity with the mouse protein was utilized (FIG. 1f). Laminin-5 is a component of hemidesmosomes, that attach basal keratinocytes to the BMZ, and is expressed uniquely by basal keratinocytes. Dark brown staining was found along the BMZ, confirming a normal morphology of the BMZ in the reconstituted skin.

As noted in FIG. 1a, in some instances the reconstitutions showed incomplete separation of the epidermal and dermal layers. A section having such non-discrete layers was stained with the laminin-5 antibody. In this thin section, pockets of keratinocytes were found in the dermis. Interestingly, laminin-5 staining was observed at the interface of the keratinocyte inclusion bodies with the dermal fibroblasts, as well as in the expected location along the BMZ. Apparently cell-cell contact between keratinocytes and fibroblasts is sufficient to trigger laminin-5 expression, and thus perhaps determines the basal character of keratinocytes.

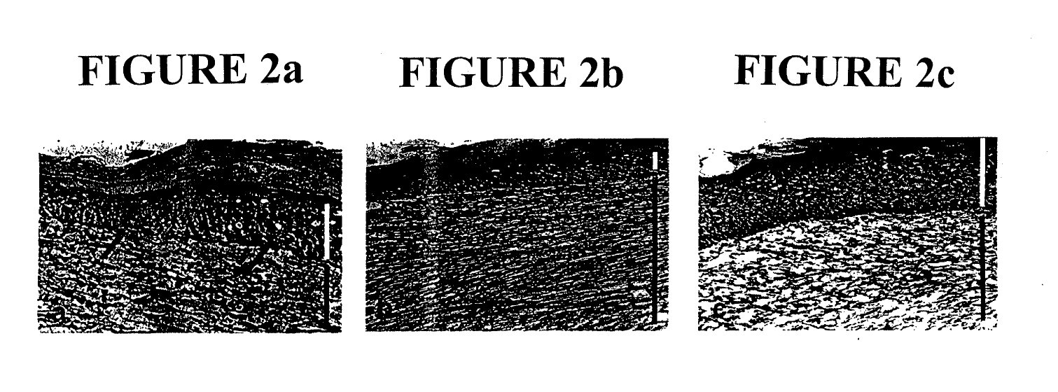

To document that the reconstituted dermis is made up of human fibroblasts, and that these cells are expressing normal markers, thin sections were stained with antibodies specific for dermal fibroblasts. Immunoperoxidase staining using the monoclonal antibody LH 7.2 that recognizes human collagen VII, was conducted (FIG. 2a). Normally, collagen VII deposition occurs just below the BMZ, and is considered to be contained at the top of the dermis, being expressed primarily by dermal fibroblasts. Collagen VII forms the anchoring fibrils, that serve as dermal attachments for hemidesmosomal components. The observed staining near the dermal-epidermal junction is of the expected normal pattern.

A marker more uniformly expressed by dermal fibroblasts is vimentin. Staining using an antibody to this marker showed ubiquitous expression in the reconstituted dermis (FIG. 2b). Another monoclonal antibody commercially marketed as specific to human fibroblasts that will not recognize mouse cells is termed 5B5, (the exact antigen is unknown). This reagent stained the reconstituted human dermis, indicating a human dermal layer beneath the human epidermis. Also shown in this particular section is a lack of staining of another cell layer located below the reconstituted dermis, probably composed of mouse fibroblasts (FIG. 2c, indicated by the grey bar). The presence of this nonstaining layer was variable in different reconstitutions, and was often absent. These human skin reconstitutions, termed skin equivalents herein, are thus, capable of recapitulating many aspects of natural full-thickness human skin.

### Example 2

### Reconstitution of a Human Genodermatosis

In the genetic skin blistering disease junctional epidermolysis bullosa (JEB) a defect in one of the genes coding for hemidesmosome proteins expressed by the basal keratinocytes is defective, see, e.g., Fine, et al., J. Am. Acad. Dermatol., 24:119 (1991). Laminin-5 is the primary component of anchoring filaments, that are crucial to hemidesmosome function, and is the site of the most common primary genetic lesion in JEB. Laminin-5 staining along the BMZ is observed when normal immortalized keratinocytes were added to the silicone chamber, biopsied after 2 weeks, cryosectioned, and immunofluorescence was conducted for laminin-5. Immunofluorescence staining was conducted with a monoclonal antibody specific for the laminin-5 β3 chain (K140).

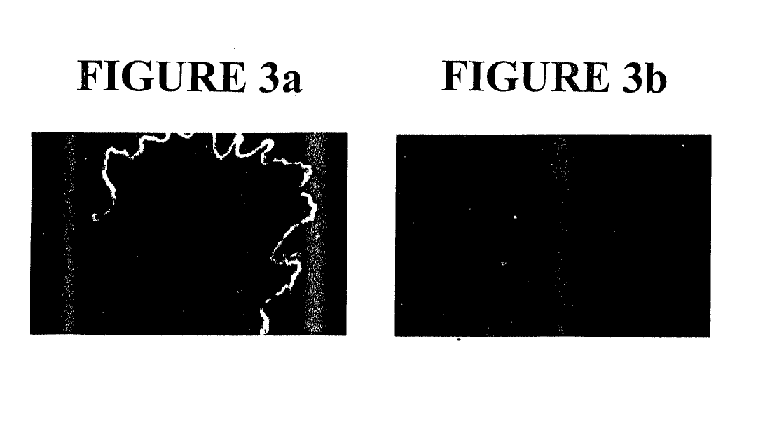

Positive laminin-5 staining is observed along the BMZ, seen as a bright undulating line, when normal immortalized keratinocytes are used in the reconstitution (FIG. 3a). In contrast, no staining is seen in a reconstitution containing JEB patient immortalized keratinocytes (FIG. 3b). Additional staining of the JEB reconstituted skin with other keratinocyte and fibroblast markers showed a normal pattern. This experiment illustrates that genetic skin diseases can be modeled using the individual skin cell type that expresses the primary genetic lesion, in this case JEB keratinocytes.

Even in skin diseases where the evidence for a genetic component is strong, but the genetic lesion has not yet been identified, the reconstitutions are informative. Often in these cases the cell type expressing the mutant gene is also unknown. By conducting mixed reconstitutions, alternatively testing patient derived keratinocyte or fibroblast populations, and matching them with cells from a normal source, the identity of the cell type needed to recapitulate the disease could be found.

### Example 3

### Targeting Expression to Epidermal or Dermal Layers

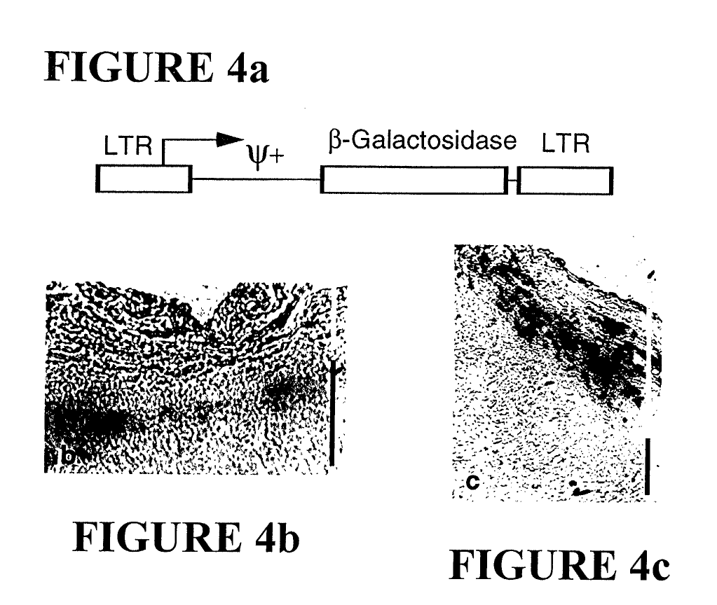

A β-galactosidase expressing retrovirus was used to infect keratinocytes or fibroblasts used in the skin equivalent (FIG. 4a). Primary fibroblasts, or primary keratinocytes were infected with amphotrophic retrovirus produced in the Phoenix helper cell line ΦNX-A, and used to reconstitute human skin. β-galactosidase staining is shown two weeks after the cells were seeded into the chambers placed on the backs of SCID mice.

As predicted, β-galactosidase staining was confined to dermal (FIG. 4b) or epidermal layers (FIG. 4c) when fibroblasts or keratinocytes, respectively, were used. These experiments again confirm that the human cells are reconstituting skin, rather than mouse cells, since only the human cells were infected with retrovirus expressing β-galactosidase. Targeted efficient expression of exogenously added genes to a single cell type is another aspect of the usefulness of this model, since the gene introduction steps can be conducted while the component cells are still in tissue culture.

### Example 4

### Reconstitutions Using Senescent Fibroblasts Recapitulate Aging Phenotype

There are multiple morphological alterations in the skin during aging. For example, the thickness of the dermis declines, collagen bundles and elastin fibrils in the dermis become more disorganized, the turnover rate of keratinocytes decreases, and fragility of the skin along the dermal-epidermal junction increases. In this application of the cell sorted skin equivalent model, addressed is the question of what aspects of the aging skin are due to senescence of dermal fibroblasts, versus strictly time dependent effects, such as changes in properties of the plasma membrane and cumulative damage to the DNA. The experiments evaluated the contribution of fibroblast cell senescence in producing an aging phenotype in the reconstituted skin.

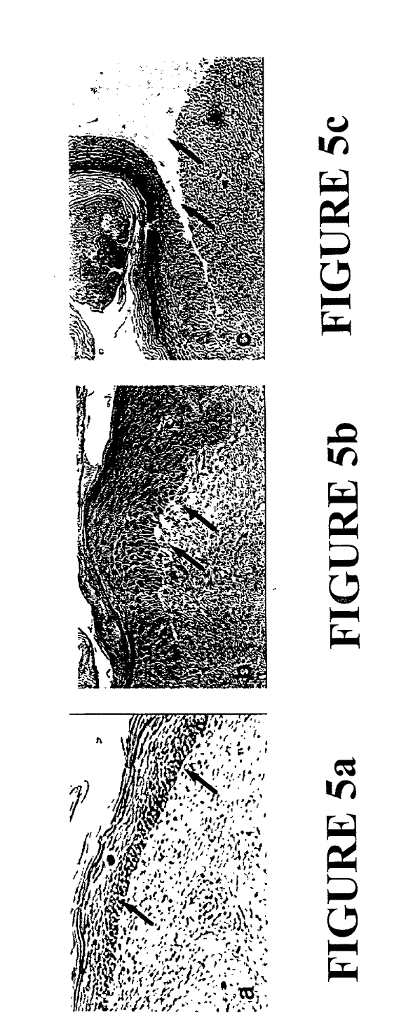

Early, middle, and late passage dermal fibroblasts were used in forming the skin equivalent. The late passage fibroblasts were grown to replicative senescence, approximately passage 80. Hemotoxylin/eosin (H/E) staining of the skin equivalent biopsies for each sample were evaluated for morphological differences. The morphology of the CSSE made, with early passage fibroblasts was normal, as expected (FIG. 5a). However, when the fibroblasts used had been passaged approximately an additional 40 doublings, some shearing along the dermal-epidermal interface was observed (FIG. 5b). The fragility along dermal-epidermal junction was the most pronounced when senescent fibroblasts, at approximately passage 80 ) were used (FIG. 5c). These experiments suggest that at least this one aspect of fragility in the skin of the elderly may be contributed by the presence of senescencing fibroblasts.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

What is claimed is:

1. A mammalian skin equivalent comprising discrete epidermal and dermal layers, wherein said dermal layer comprises fibroblasts and said epidermal layer comprises differentiated keratinocytes and basal keratinocytes, wherein said basal keratinocytes are aligned in a layer in direct contact with said dermal layer.

2. The skin equivalent of claim 1 wherein said skin equivalent lacks at least one of melanocytes, hair follicles, sweat glands and nerve endings.

3. The skin equivalent of claim 1 wherein said fibroblasts and keratinocytes have different genotypes.

4. The skin equivalent of claim 1 wherein at least one of said fibroblasts or keratinocytes are abnormal.

5. The skin equivalent of claim 1 wherein at least one of said fibroblasts or keratinocytes are immortalized.

6. The skin equivalent of claim 1 wherein at least one of said fibroblasts or keratinocytes are genetically engineered.

7. A mammalian skin equivalent comprising discrete epidermal and dermal layers wherein said dermal layer comprises fibroblasts and said epidermal layer comprises keratinocytes and wherein at least one of said fibroblasts or keratinocytes are abnormal.

8. The skin equivalent of claim 7 wherein said skin equivalent lacks at least one of melanocytes, hair follicles, sweat glands and nerve endings.

9. The skin equivalent of claim 7 wherein said fibroblasts and keratinocytes have different genotypes.

10. The skin equivalent of claim 7 wherein at least one of said fibroblasts or keratinocytes are immortalized.

11. The skin equivalent of claim 7 wherein at least one of said abnormal fibroblasts or keratinocytes are genetically engineered.

12. A mammalian skin equivalent comprising discrete epidermal and dermal layers wherein said dermal layer comprises fibroblasts and said epidermal layer comprises keratinocytes and wherein at least one of said fibroblasts or keratinocytes are senescencing.

13. The skin equivalent of claim 12 wherein said skin equivalent lacks at least one of melanocytes, hair follicles, sweat glands and nerve endings.

14. The skin equivalent of claim 12 wherein said fibroblasts and keratinocytes have different genotypes.

15. The skin equivalent of claim 12 wherein at least one of said abnormal fibroblasts or keratinocytes are genetically engineered.

16. A method for forming a mammalian skin equivalent having discrete dermal and epidermal layers comprising:
providing a mixture comprising keratinocytes and fibroblasts and
allowing said keratinocytes and fibroblasts to sort to form a mammalian skin equivalent having discrete dermal and epidermal layers.

17. The method of claim 16 wherein said fibroblasts or keratinocytes are abnormal.

18. The method of claim 16 wherein said abnormal fibroblasts or keratinocytes are genetically engineered.

19. The method of claim 16 wherein said fibroblasts are senescencing fibroblasts.

20. The skin equivalent made according to claim 16.

21. An animal model comprising the skin equivalent made according to claim 16.

22. An assay for identifying a candidate agent having an affect on skin comprising:
providing the skin equivalent made according to claim 16;
contacting said skin equivalent with a candidate agent;
determining whether said candidate agent has an affect on said skin equivalent as an indication of the affect of said candidate agent on said skin.

23. The assay of claim 22 wherein said skin equivalent is abnormal.

24. The assay of claim 22 wherein said skin equivalent is aged.

25. The assay of claim 22 wherein said candidate agent is selected from said group consisting of cosmetics, pharmaceuticals, cells, nucleic acids, factors and peptides.

26. An assay for identifying a candidate agent having an affect on skin comprising:
providing keratinocytes and fibroblasts;
adding a candidate agent to said keratinocytes and/or fibroblasts;
mixing said keratinocytes and fibroblasts;
allowing said keratinocytes and fibroblasts to sort to form a mammalian skin equivalent;
determining whether said candidate agent has an affect on said skin equivalent as an indication of the affect of said candidate agent on said skin.

27. The assay of claim 26 wherein said candidate agent is selected from said group consisting of cosmetics, pharmaceuticals, cells, nucleic acids, factors and peptides.

28. A method of treating an individual in need of a skin graft, comprising:
providing said skin equivalent made according to claim 16 to said individual.

29. The method of claim 28 wherein said mixture is applied to tissue on said individual and said skin is formed thereon.

30. The method of claim 28 wherein said fibroblasts and/or keratinocytes are taken from said individual.
